(19) United States
(12) Patent Application Publication
(10) Pub. No.: US 2014/0275467 A1
(43) Pub. Date: Sep. 18, 2014

(54) Polylactone Polymers Prepared from Monol and Diol Polymerization Initiators Processing Two or More Carboxylic Acid Groups

(71) Applicant: Ethicon, Inc., Somerville, NJ (US)

(72) Inventors: Dennis D. Jamiolkowski, Long Valley, NJ (US); Modesto Erneta, Princeton Junction, NJ (US); Terry E. Lawler, Baldwin, GA (US); Rao S. Bezwada, Whitehouse Station, NJ (US)

(73) Assignee: Ethicon, Inc., Somerville, NJ (US)

(21) Appl. No.: 14/195,220

(22) Filed: Mar. 3, 2014

Related U.S. Application Data
(60) Provisional application No. 61/789,356, filed on Mar. 15, 2013.

(51) Int. Cl. A61L 17/10; A61L 31/06

(52) U.S. Cl. CPC A61L 17/105; A61L 31/06
USPC 528/274

(57) ABSTRACT

The present invention is directed to a novel polymerization process for making novel absorbable, linear polylactone polymers prepared using novel polymerization initiators to achieve rates of mechanical property loss or of absorption of articles made from the polymers that are at least about 1.2 times faster than the rates of mechanical property loss of polymers made by similar processes utilizing conventional initiators. The novel polymerization initiators include monols or diols possessing at least one primary alcohol group and two or more carboxylic acid groups. The invention also is directed to absorbable polylactone polymers prepared by processes of the present invention and to medical devices made from such polymers.

### FIELD OF THE INVENTION

The present invention relates to absorbable polylactone copolymers suitable for use in implantable medical devices and methods of making such copolymers, which methods include the use of mono-alcohol or di-alcohol polymerization initiators, otherwise known as molecular weight control agents, to polymers prepared by such methods and to medical devices prepared from such polymers.

### BACKGROUND OF THE INVENTION

The use of initiators such as glycolic acid has been well known in the art and science of ring opening polymerizations of lactones. It has been recognized that the alcohol group readily participates in a reaction that incorporates the initiator in the growing chain. Alcohols such as dodecanol have been used as well. Diols and polyols have also been used. It is known that including a carboxylic acid group in the initiator can increase the rate at which the polymer loses mechanical strength and can increase the rate at which it absorbs.

Homopolymers and copolymers of p-dioxanone (PDO) are known for use in the medical device and pharmaceutical fields due to their low toxicity, softness and flexibility. Poly(p-dioxanone) (PDO) homopolymer in particular has been suggested as an absorbable polymer for use in synthetic surgical devices. By the early 1980's, the PDS homopolymer was used by surgeons in the form of a monofilament surgical suture. Since that time, many p-dioxanone copolymers have been described for use in such devices. Surgical monofilament sutures based on a copolymer prepared from trimethylene carbonate (TMC), glycolide (GLY) and p-dioxanone (PDO) monomer currently are available for use. PDO based polymeric materials also can be injection molded into a number of non-filamentous surgical devices such as surgical clips and fasteners for use in, e.g., meniscal repair. These surgical articles take full advantage of the general toughness exhibited by this family of homopolymers and copolymers known heretofore.

U.S. Pat. No. 2,362,511 discloses a polyglycolide resin derived from glycolide and from about 20 to about 55 weight percent of a carboxylic acid such as lactic acid, tartaric acid, malic acid, citric acid, etc.

U.S. Pat. No. 3,169,945 discloses a homopolymer of epsilon-caprolactone obtained by polymerizing epsilon-caprolactone in the presence of a carboxylic acid initiator such as citric acid, aconitic acid, mellitic acid, pyromellitic acid, etc.

U.S. Pat. No. 3,942,532 discloses a surgical suture coating composition comprising a polyester derived from the esterification of a low molecular weight glycol and a dimeric acid such as succinic acid, glutaric acid, adipic acid, etc.

U.S. Pat. No. 4,624,256 discloses a bioabsorbable copolymer derived from at least 90 weight percent of epsilon-caprolactone and up to 10 weight percent of a carboxylic acid such as glycolic acid, lactic acid, malic acid, succinic acid, etc.

U.S. Pat. No. 4,643,191 discloses a copolymer obtained by: (1) the polymerization of p-dioxanone in the presence of a carboxylic acid initiator such as glycolic acid, lactic acid, etc., to form a mixture of p-dioxanone monomer and homopolymer and (2) subsequent polymerization of (1) with lactide to form the copolymer.

U.S. Pat. No. 5,076,807 discloses a bioabsorbable copolymer derived from polymerizing p-dioxanone and glycolide in the presence of a carboxylic acid initiator, e.g., glycolic acid or lactic acid.

Copolymers derived from epsilon-caprolactone and at least one other monomer such as lactide, glycolide, glycolic acid, p-dioxanone and trimethylene carbonate are disclosed in U.S. Pat. Nos. 4,605,730; 4,624,256; 4,700,704; 4,788,979; 4,791,929; 4,994,074; 5,076,807; 5,080,665; 5,085,629, and 5,100,433.

U.S. Pat. No. 5,425,949 describes a bioabsorbable copolymer that is obtained from the polymerization of a major amount of ε-caprolactone and a minor amount of at least one other copolymerizable monomer in the presence of an initiator possessing at least two carboxylic acid groups. The copolymer is useful, inter alia, as a coating for a surgical suture. U.S. Pat. No. 5,425,949 clearly does not, however, anticipate the need for at least one primary hydroxyl group. Col 2. lines 9 to 13 describe as suitable carboxylic acid initiators as including succinic acid, maleic acid, glutaric acid, adipic acid, pimelic acid, suberic acid, azelaic acid, sebacic acid, malic acid, tartaric acid, citric acid, aconitic acid, pyromellitic acid, mellitic acid, etc., and combinations thereof. It should be noted that although citric acid, 2-hydroxy propane-1,2,3-tricarboxylic acid, possesses three carboxylic acid groups, its single alcohol group is tertiary in nature.

Polymers having units derived from citric acid have been described elsewhere for various purposes. For example, U.S. Pat. No. 3,661,955 discloses polyesters of citric acid and sorbitol useful as intermediates in the manufacture of medicine, emulsifiers and as additives to yeast raised products. As another example, U.S. Pat. No. 5,026,821 discloses hydrophilic polymers composed of polyamides resulting from the condensation of citric acid with diamines. The polymers are employed as carriers or reservoirs for the controlled release of drugs, as sutures, surgical prostheses, and surgical adhesives.

U.S. Pat. No. 5,480,963 describes bioabsorbable copolymers that are derived from tricarboxylic acids and triols. This patent is not directed towards linear polymers. U.S. Pat. No. 5,480,963 is directed towards cross-linked products.

Segmental block copolymers composed of p-dioxanone and glycolide (at a molar ratio of PDO:GLY of approximately 90:10) were thought to be polymers potentially suitable for use as a “soft” monofilament suture having a breaking strength retention (BSR) profile similar to Vicryl® sutures available from Ethicon, Inc. However, advantageous as these copolymers are, it is known that they absorb in the body at a certain rate, limiting their utility as “soft” monofilament sutures in surgical applications in which rapid degradation is desirable.

There is a long felt need in this art for novel absorbable polymers having utility in “soft” monofilament sutures and methods for making such polymers. It would be advantageous, then, to provide novel polymerization processes necessary to produce such polymers having properties suitable for conversion to “soft” monofilament sutures, as well as other absorbable polymers of a variety of “softness” including “hard” polymers, which lose their mechanical properties quickly with a corresponding rapid absorption, as well as other implantable medical devices. The present invention provides such processes, polymers made by such processes and having unique properties, and medical devices, including sutures made from such polymers. There is additionally a need for a polymerization process that proceeds smoothly, quickly and reliably to completion.

### SUMMARY OF THE INVENTION

Accordingly, a novel process utilizing preferred initiators that result in novel linear polymers with increased rates of mechanical property loss and increased rates of absorption is provided. The novel process of the present invention is directed to a polymerization process for making absorbable polylactone polymers, wherein a lactone monomer comprising glycolide, L(−)-lactide, D(+)-lactide, meso-lactide, 1,4-dioxanone, ε-caprolactone, or trimethylenecarbonate is contacted with a polymerization initiator comprising a mono-alcohol containing a primary hydroxyl group and having two or more carboxylic acid groups or alternately a di-alcohol containing at least one primary alcohol group and having two or more carboxylic acid groups. The polymerization initiator is present at a molar ratio of lactone monomer to initiator ranging from about 300:1 to about 50,000:1. The process takes place in the presence of a catalyst under conditions sufficient to effectively polymerize the monomers, thereby providing the novel absorbable linear polylactone polymers. Suitable catalysts include many organotin compounds. When medical devices are manufactured from certain polymers prepared by the novel processes of the present strength, the loss of strength or the rate of absorption is at least about 1.2 times faster, and preferably greater than about 1.5 times faster, than the loss of strength or the rate of absorption of medical devices made from polylactone polymers made by a substantially similar or the same polymerization process, but utilizing either monol or diol initiators which do not contain at least two carboxylic acid groups.

The present invention also is directed to absorbable polylactone polymers prepared by processes of the present invention and to medical devices comprising such polymers.

Another aspect of the present invention is a substantially linear aliphatic absorbable polyester comprising a monovalent unit of formula I:

—O—CH₂—R₁ I

and divalent repeating units selected from the group of formulae consisting of:

[—O—CH₂—C(O)—]ₐ II

[—O—C(H)(CH₃)—C(O)—]_{b} III

[—O—CH₂CH₂—O—CH₂CH₂—C(O)—]_{c} IV

[—O—CH₂CH₂CH₂CH₂CH₂—C(O)—]_{d} V

[—O—CH₂CH₂CH₂—O—C(O)—]ₑ VI

and combinations thereof, wherein R₁ is an alkyl group containing two or more carboxylic acid groups, and a, b, c, d, and e are integers such that the weight average molecular weight of said substantially linear aliphatic absorbable polyester is between about 35,000 Daltons and 200,000 Daltons.

Yet another aspect of the present invention is a substantially linear aliphatic absorbable polyester comprising a first divalent unit of formula IA:

[—O—C(R₂)(R₃)—R₄—CH₂—O—] IA

and divalent repeating units selected from the group of formulae consisting of:

[—O—CH₂—C(O)—]ₐ IIA

[—O—C(H)(CH₃)—C(O)—]_{b} IIIA

[—O—CH₂CH₂—O—CH₂CH₂—C(O)—]_{c} IVA

[—O—CH₂CH₂CH₂CH₂CH₂—C(O)—]_{d} VA

[—O—CH₂CH₂CH₂—O—C(O)—]ₑ VIA

and combinations thereof, wherein R₂ and R₃ are independently hydrogen or an alkyl group containing 1 to 8 carbon atoms, R₄ is an alkyl group containing two or more carboxylic acid groups, and a, b, c, d, and e are integers such that the weight average molecular weight of said substantially linear aliphatic absorbable polyester is between about 35,000 Daltons and 200,000 Daltons.

Still yet another aspect of the present invention is a novel, linear absorbable polymer made by the novel process of the present invention.

Yet another aspect of the present invention is a medical device made from a novel polymer of the present invention.

A further aspect of the present invention is an absorbable suture made from a novel polymer of the present invention, in particular, a surgical suture.

Thus, the present invention provides novel medical devices made from the novel absorbable linear polymers having increased loss of strength or rate of absorption as compared to absorbable polymers made by conventional processing, as taken under the same or similar measurement conditions or techniques. Preferred initiators are provided that result in linear polymers with increased rates of mechanical property loss and increased rates of absorption.

These and other advantages and characteristics of the present invention will become more apparent from the following description and accompanying drawings.

### BRIEF DESCRIPTION OF THE DRAWINGS

FIG. 1 is a plot of unreacted ε-caprolactone monomer versus the reaction time for the polymerizations of Examples 1 and 2.

### DETAILED DESCRIPTION OF THE INVENTION

This invention is directed towards the production of substantially linear polymers as opposed to star-shaped materials. The invention is further directed towards medical devices, especially surgical devices, especially fibers and sutures.

Properties of monofilament fibers produced from polylactone polymers, including breaking strength retention profiles and absorption times, are found to vary depending on whether the polymerization reactions used to prepare the polymers were initiated with monol or diol initiators that contain at least one primary alcohol group and contain two or more carboxylic acid groups, as compared to monol or diol initiators not containing at least two carboxylic acid groups. It has been discovered, surprisingly and unexpectedly, that the use of such polymerization initiators having at least two carboxylic acid groups in polymerization processes, e.g., ring-opening polymerization, may provide certain linear polylactone polymers that, in turn, provide articles of manufacture that exhibit advantageous breaking strength retention profiles and absorption times.

In order to produce a high molecular weight polymer by a ring-opening polymerization (ROP) in a timely, reproducible and economical fashion, a catalyst usually is combined with a mono- or multi-functional initiator. These initiators are desirably hydroxyl-containing compounds, most preferably primary alcohols that can be used to generate linear or branched polymers. If the initiator contains one or two hydroxyl groups, a linear material will result. It is expected that both mono- and di-functional initiators normally will produce linear materials because one chain, without branch points, is produced from each molecule of initiator. If the initiator contains three or more hydroxyls, branched materials are generally formed. Various conventional catalysts for the ring ring-opening polymerization of lactones are known and have been used. They are generally metal-based and include the organic titanates and zirconates (as sold by DuPont under the tradename TYZOR). Organotin compounds have found great utility as catalysts for the ring-opening polymerization of lactones for medical applications. Tin catalysts include Sn (IV) compounds such as dibutyltin oxide and Sn (II) compounds such as stannous chloride. Particularly advantageous for use as a catalyst is stannous octoate.

The polymerization is conveniently done in a conventional bulk process, i.e., solventless, although it also may be conducted in solution. The polymerization is typically conducted in the melt, that is, above the melting points of the various monomers making up the feed, as well as above the melting point of the forming polymer. In some special cases, the ring-opening polymerization of certain lactones can be conducted in the solid state, that is, below the melting point of the forming polymer. An example of the latter is the homopolymerization of p-dioxanone. Although the total-monomer-to-total-initiator molar ratio can typically range from about 300:1 to about 50,000:1, the preferred range of the total-monomer-to-total-initiator molar ratio for polymer to be used in extrusion and injection molding processes ranges from about 400:1 to about 2,000:1. This is because the amount of initiator greatly influences the molecular weight of the formed resin. In the absence of side reactions, each initiator molecule ideally generates one polymer chain. The more relative initiator available, the greater the number of chains formed and consequently the lower the molecular weight of the resin formed. In the preferred range of total-monomer-to-total-initiator molar ratio of about 400:1 to about 2,000:1, the molecular weight of the resulting polymer is more suitable for extrusion and injection molding applications.

Cyclic esters, i.e., lactones, that function as suitable monomers can be selected from the group comprising small rings, especially the 5-, 6-, and 7-member rings. Of particular utility are the lactones containing a heteroatom, especially oxygen, adjacent to the α-carbon. Preferred 6-member cyclic esters include glycolide, L(−)-lactide, D(+)-lactide, meso-lactide, and p-dioxanone. Trimethylene carbonate is a preferred monomer. A preferred 7-member lactone is ε-caprolactone (epsilon-caprolactone). The characteristics of suitable monomer for the present invention include those that provide reasonable, sufficiently effective reaction rates under suitable reaction conditions. The polymers that are formed are advantageously biocompatible, making them suitable for the fabrication of medical devices.

One of the characteristic methods for preparation of branched and highly functional aliphatic polyesters might involve hydroxyl functionalities as the pendant groups in a polymer chain. See for instance the work of M. Trollsas, J. L. Hedrick, D. Mecerreyes, Ph. Dubois, R. Jerome, H. Ihre, and A. Hult, in Macromolecules (1998), 31, 2756. These molecules containing a plurality of pendant hydroxyl groups might serve as macroinitiators for the initiation of ring-containing monomers in a subsequent copolymerization step to prepare dendri-graft (comb) molecular structures. Similarly, hydroxyl groups of multifunctional initiators might be fully substituted to produce star-shaped polymers with two, four, five and six arms. See for instance the work of A. Schindler, Y. M. Hibionada, and C. G. Pitt in the Journal of Polymer Science: Polymer Chemistry Edition (1982), 20, 319 as well as the work of C. A. P Joziasse, H. Grablowitz, and A. J. Pennings in Macromol. Chem. Phys. (2000), 201, 107.

Due to their unique molecular architecture, branched compounds exhibit different physiochemical properties compared to their linear counterparts. It is generally recognized that long-branches can decrease viscosity, thus improving processability in some instances, and increase elasticity, while short chain branches predominately affect crystallinity. For instance, F. Tasaka, Y. Ohya, and T. Ouchi, in Macromolecules (2001), 34, 5494), disclose graft polymerized 1-lactide (LA) in bulk using Sn(Oct)₂ in the presence of poly[(Glc-Ser)-LA] having pendant hydroxyl groups as a macroinitiator. Such obtained comb-like polymers showed a substantial reduction in crystallinity compared to the linear poly (L-lactide), PLLA (15-22% vs. 55%). An abrupt decrease in both the glass transition temperature (40-43° C. vs. 65° C.) and the melting point (135-140° C. vs. 167° C.) was also detected. Owing to the lower crystallinity, biological properties are affected as well. In vitro degradation rate of comb-type PLLA was found to be significantly faster than that of linear PLLA. The novel polymers of the present invention are substantially linear in nature and are not branched resins.

The rheology of a polymer melt even within one structure or chemistry, as related to processing and fabrication, is affected by many factors such as the molecular weight and molecular weight distribution, the polymer architecture and blending. In particular, long chain branching has a significant contribution. Although limiting the synthesis to linear materials helps to simplify the processing and fabrication issues that would arise because of the contributions that branching would bring to the melt rheology, crystallization concerns speak away from branched materials. Although we do not wish to be limited by scientific theory, branched polymers frequently are more difficult to crystallize when compared to unbranched (linear) polymers of the material. They are thus less suited to the formation of certain medical devices.

Returning to linear materials, mono- or di-functional initiators, as already described, have found extensive use in producing polymers useful for producing absorbable surgical devices. Diols have been used in ring opening “pre-polymerizations” to produce α,ω-dihydroxymacroinitiators (alpha, omega-dihydroxymacroinitiators) that are then used in a subsequent copolymerization to produce polymers with special sequence distributions. This sequential addition ring opening polymerization (ROP), in which a monomer feed portion is added in a subsequent step, is one method to make so-called segmented block copolyesters. An example is a commercially available glycolide/epsilon-caprolactone copolymer that has enjoyed considerable commercial success. See R. S. Bezwada, D. D. Jamiolkowski, et. al., “MONOCRYL™ Suture, a New Ultra-Pliable Absorbable Monofilament Suture”, Biomaterials, 16 (15), 1141-1148 (1995).

Within the scope of the present invention is the use of mono- or di-functional initiators in sequential addition ring opening polymerizations in which the monomer feed is added in sequence. That is, portions of the total monomer are allowed to enter the reactor in sequence or in multiple steps, as opposed to having all of the monomer added at once. It is also within the scope of the present invention to have polymerization processes in which the monomer is indeed added to the reactor in substantially a single step at the start of the polymerization. In all cases, it should be understood that the monomers employed can be added to the reactor as solids, in the case where the monomers are indeed a solid at room temperature, or added as molten liquids. If the reaction is to be conducted in the presence of a solvent, the monomers may be added in solution. It is also within the scope of the present invention polymerization processes in which the initiators are added in sequence or are added independently as a function of time.

Initiators of lactone ring-opening polymerizations can, under the right conditions, be aliphatic alcohols, phenols, thiols or mercaptans, thiophenols, or amines. Alcohols, of course, possess hydroxyl groups, while thiols possess sulfhydryl groups. The alcohols and amines may be primary, secondary or tertiary and they may be linear or branched. Of particular utility are aliphatic alcohols, especially primary aliphatic alcohols. Of even greater utility are primary aliphatic alcohols of low volatility. Once placed in the reactor, such initiators are not easily lost during vacuum purging cycles, thus allowing much better process control of the resulting polymer's molecular weight. For purposes of the present invention, in determining whether an initiator is classified as a monol or a diol initiator, one need only determine the number of hydroxy groups present in the compound. If an initiator contains one hydroxyl group it is classified as a monol; if it contains two hydroxyl groups it is classified as a diol. Although an initiator may be a monol or a diol initiator, it may simultaneously contain a carboxylic acid groups. The subject of the present invention are those monol and diol initiators that contain at least one primary alcohol group and simultaneously contain at least two carboxylic acid groups.

The monol initiators useful in the practice of the present invention are compounds that contain one primary hydroxyl group and simultaneously contain at least two carboxylic acid groups. Examples of inventive primary monol dicarboxylic acids include: C₄H₆O₅, HOOC—CH(CH₂OH)—COOH; C₅H₈O₅, HOOC—C(CH₃)(CH₂OH)—COOH; C₇H₁₂O₅, HOOCCH₂—C(CH₃)(CH₂OH)—CH₂COOH; and C₉H₁₆O₅, HOOCCH₂—C(CH₂CH₃)(CH₂CH₂OH)—CH₂COOH. An example of a preferred monol initiator of the subject invention is 1-hydroxy-2,2,2-ethanetricarboxylic acid, also known as 1-hydroxy-2,2,2-trimethcarboxyethane.

The diol initiators of the present invention are compounds that contain two hydroxyl groups, at least one of which is primary in nature and simultaneously contain at least two carboxylic acid groups. The hydroxyl groups of the most preferred diol initiators of the subject invention are both primary in nature. Examples of inventive diol dicarboxylic acids, with at least one of the alcohol groups primary in nature include: C₅H₈O₆, HOOC—C(CH₂OH)₂—COOH; C₇H₁₂O₆, HOOCCH₂—C(CH₂OH)₂—CH₂COOH; C₈H₁₄O₆, HOOCCH₂—C(CH₂OH)(CH₂CH₂OH)—CH₂COOH; and C₉H₁₆O₆, HOOCCH₂—C(CH₂CH₂OH)₂—CH₂COOH. An example of a preferred diol initiator of the subject invention is 1,3-dihydroxy-2,2-dicarboxypropane (also known as 2,2-dimethylol-malonic acid).

Examples of inventive primary monol tricarboxylic acids include: C₅H₆O₇, HOCH₂—C—(COOH)₃; C₇H₁₀O₇, HOCH₂—C(CH₂COOH)₂—COOH; C₈H₁₂O₇, HOCH₂—C—(CH₂COOH)₃; and C₉H₁₄O₇, HOCH₂CH₂—C—(CH₂COOH)₃.

Examples of non-inventive diol monocarboxylic acids with at least one of the alcohol groups primary in nature include: C₅H₁₀O₄, HOCH₂—C(CH₃)(COOH)—CH₂OH; and C₆H₁₂O₄, HOCH₂—C(CH₃)(CH₂COOH)—CH₂OH; these latter two compounds are non-inventive because they possess only one carboxylic acid group.

It may be necessary for various reasons to determine the composition of the formed polymers. The use of NMR (nuclear magnetic resonance) in elucidating structure is well known. Because the amounts of initiator are relatively small, it may be difficult to identify what initiators were employed in the polymerization. One convenient way to do so, however, is to completely hydrolyze the polyester concurrently converting the initiator moiety back to the corresponding original free initiator. For example, a p-dioxanone glycolide copolymer initiated with dodecanol and diethylene glycol would have the alcohols converted to esters in the course of the polymerization. Hydrolysis of the polyester would result in the generation of 2-hydroxyethoxyglycolic acid (ring opened form of p-dioxanone), glycolic acid, dodecanol and diethylene glycol. The composition can then be determined by analyzing the hydrolyzate by a suitable means. These include LC (liquid chromatographic) methods.

The novel absorbable polymers of the present invention are substantially linear aliphatic polyesters having weight average molecular weights between about 35,000 Daltons and 200,000 Daltons. The corresponding number average molecular weights of the novel absorbable polymers of the present invention range from about 17,000 Daltons to about 100,000 Daltons. The compositions of the inventive absorbable polymers may vary widely but are typically based on repeat units derived from the polymerization of glycolide, p-dioxanone, L(−)-lactide, D (+)-lactide, meso-lactide, ε-caprolactone and trimethylene carbonate in any combination. Of particular utility for some surgical applications are those absorbable polymers of the present invention that have the ability to crystallize; of these, crystallinity ranges from about 10% to about 45% may be particularly useful. The melt viscosities exhibited by the absorbable polymers of the present invention must be high enough to support preferred manufacturing techniques such as melt extrusion in the case of fiber formation; they may not be so high as to lose the ability to be formed into useful articles.

The substantially linear aliphatic absorbable polyester polymers of the present invention made using the novel monols of the present invention will consist of a monovalent unit of formula I:

—O—CH₂—R₁ I

and divalent repeating units selected from the group of formulae consisting of:

[—O—CH₂—C(O)—]ₐ II

[—O—C(H)(CH₃)—C(O)—]_{b} III

[—O—CH₂CH₂—O—CH₂CH₂—C(O)—]_{c} IV

[—O—CH₂CH₂CH₂CH₂CH₂—C(O)—]_{d} V

[—O—CH₂CH₂CH₂—O—C(O)—]ₑ VI

and combinations thereof, wherein R₁ is an alkyl group containing two or more carboxylic acid groups, and a, b, c, d, and e are integers such that the weight average molecular weight of said substantially linear aliphatic absorbable polyester is between about 35,000 Daltons and 200,000 Daltons.

The substantially linear aliphatic absorbable polyester polymers of the present invention made using the novel diols of the present invention will consist of a first divalent unit of formula IA:

[—O—C(R₂)(R₃)—R₄—CH₂—O—] IA

and divalent repeating units selected from the group of formulae consisting of:

[—O—CH₂—C(O)—]ₐ IIA

[—O—C(H)(CH₃)—C(O)—]_{b} IIIA

[—O—CH₂CH₂—O—CH₂CH₂—C(O)—]_{c} IVA

[—O—CH₂CH₂CH₂CH₂CH₂—C(O)—]_{d} VA

[—O—CH₂CH₂CH₂—O—C(O)—]ₑ VIA

and combinations thereof, wherein R₂ and R₃ are independently hydrogen or an alkyl group containing 1 to 8 carbon atoms, R₄ is an alkyl group containing two or more carboxylic acid groups, and a, b, c, d, and e are integers such that the weight average molecular weight of said substantially linear aliphatic absorbable polyester is between about 35,000 Daltons and 200,000 Daltons.

The novel absorbable polymers of the present invention manufactured using the process of the present invention may be used in a variety of conventional medical devices including sutures of the traditional variety and sutures of the barbed variety, monofilament fibers, multifilament yarn fibers, meshes, clips, staples, fixation devices of various designs, mechanically strong films, adhesion prevention devices and equivalents thereof. The medical devices may be manufactured using various conventional processes including melt extrusion, solution spinning, drawing, injection molding, melt blowing, rotomolding, and the like.

The following examples are illustrative of the principles and practice of the present invention, although not limited thereto.

### Example 1

### L-Tartaric Acid as Initiator in the Synthesis of an ABA-Type Block Copolymer of ε-Caprolactone and Glycolide

This example shows that lactone polymerization reactions catalyzed by tin compounds, such as stannous octoate, that are initiated by secondary alcohol initiators, exhibit lower polymerization reaction rates than the reactions initiated by primary alcohol initiators, such as diethylene glycol (DEG). A typical DEG initiated polymerization is shown in Example 2.

Of particular interest are initiators containing carboxylic acid groups that we have found enhance the rate of hydrolysis of lactone type polymers synthesized therefrom. The secondary alcohol initiator of this example is L-tartaric acid. As seen herein, at equal reaction times, initiators that contain secondary alcohol groups lead to a lower conversion of ε-caprolactone monomer into polymer than the primary alcohol type initiators, such as diethylene glycol (DEG).

For the ABA copolymer prepared in this example, “B” represents a randomized mid-block of 45/55 mole ratio of ε-caprolactone/glycolide and “A” represents polymerized glycolide (PGA) blocks. The intended overall composition of the copolymer is 25 mole % ε-caprolactone and 75 mole % PGA. The method of preparation was a two-stage polymerization in which the mid-block composition was prepared first and additional glycolide monomer was added in a subsequent step. Although these types of polymers are frequently denoted as ABA copolymers, the sequence distribution of the repeat units may often not exhibit a strictly ABA structure, as transesterification and other side reactions may cause sequence errors.

Into a conventional two gallon reactor provided with stirrer and jacket with heating medium was charged 1,481.2 grams (12.977 moles) of ε-caprolactone, 1,841 grams (15.861 moles) of glycolide, 7.67 grams (0.03999 moles) of L-tartaric acid and 3.92 mls of a 0.33 molar solution of stannous octoate in toluene. In this example, the molar ratio of all monomers (including the glycolide subsequently added in the second stage of the polymerization) to catalyst was 40,000:1. Into a separate conventional melt tank was charged 2,677.8 grams (23.07 moles) of glycolide (second stage). The reactor and the melt tank were kept under 1 mm Hg vacuum for 20 minutes and the vacuum was released with nitrogen. The vacuum and nitrogen-breaking step was repeated. The reactor contents were heated by means of fluid circulation through the reactor jacket until the batch temperature reached 180° C., which took about one hour. This was designated as “0” time. The reaction was continued for 6 additional hours at a heating fluid temperature of about 197° C. At this point, the “second stage” glycolide that has been previously melted in the melt tank was added to the reactor. The reaction was continued at an approximate heating fluid temperature of 203° C. for 80 minutes. The product was “dropped” or discharged and cooled. The formed resin can be pelletized upon discharge by methods such as strand pelletization or the cooled discharged resin can be ground and sieved. The divided resin was dried in a tumble drier at room temperature and vacuum for 18 hours, followed by heating under vacuum for 24 hours; the dried resin was allowed to cool and was stored under vacuum.

During the course of the reaction, samples were taken from the reactor and were analyzed by NMR spectroscopy for monomer and polymer composition. Samples were designated as hours after “0” time (0+x) or as minutes after the second-stage glycolide transfer (T+y) The compositions are given on a molar basis. The results are displayed in Table I.

TABLE I

| Sample | PGA | GLY | PCL | Cap |
|---|---|---|---|---|
| 0 + 1 | 56.6 | 0.3 | 18.4 | 24.7 |
| 0 + 2 | 55.8 | 0.4 | 21.3 | 22.5 |
| 0 + 3 | 56.1 | 0.4 | 23.8 | 19.8 |
| 0 + 4 | 58.1 | 0.0 | 25.8 | 16.1 |
| 0 + 5 | 55.7 | 0.1 | 33.4 | 10.8 |
| 0 + 6 | 55.5 | 0.2 | 33.8 | 10.5 |
| T + 65 | 75.1 | 0.6 | 19.4 | 4.8 |
| Drop (T + 80) | 75.1 | 0.5 | 19.9 | 4.5 |
| Dried | 80.4 | 0.1 | 19.5 | 0.0 |

Wherein PGA refers to polymerized glycolide, GLY refers to (unpolymerized or otherwise free) glycolide monomer, PCL refers to polymerized caprolactone, and CAP refers to (unpolymerized or otherwise free) caprolactone monomer.

### Example 2

### Diethylene Glycol (DEG) as Initiator in the Synthesis of an ABA-Type Block Copolymer of ε-Caprolactone and Glycolide

This example focuses on the synthesis of block “B and provides reaction rate data to be compared to the tartaric acid initiation data generated in Example 1.

Into a two gallon reactor provided with stirrer and jacket with heating medium was charged 1,234.3 grams (10.814 moles) of ε-caprolactone, 1,534.2 grams (13.217 moles) of glycolide, 3.531 grams (0.0332744 moles) of diethylene glycol (DEG) and 2.38 mls of a 0.33 molar solution of stannous octoate in toluene. In this example the molar ratio of all monomers (including the glycolide subsequently added in the second stage of the polymerization) to catalyst was 55,000:1. Into a separate melt tank was charged 2,231.5 grams (19.225 moles) of glycolide (second stage). The reactor and the melt tank were kept under 1 mm hg vacuum for 20 minutes and the vacuum was released with nitrogen. The vacuum and nitrogen-breaking step was repeated. The reactor contents were heated by means of fluid circulation through the reactor jacket until the batch temperature reached 180° C. in about one hour. This was designated as “0” time. The reaction was continued for 6 additional hours at a heating fluid temperature of about 197° C. At this point glycolide that had been previously melted in a separate melt tank was added to the reactor at a controlled rate. The reaction was continued at an approximate heating fluid temperature of 203° C. for 75 minutes and the product was discharged, cooled and dried in a tumble drier at room temperature and vacuum for 18 hours, followed by heating under vacuum for 24 hours and cooling.

During the course of the reaction, samples were taken from the reactor and were analyzed by NMR for monomer and polymer composition. Samples were designated as hours after “0” time. Data is provided for the first stage polymerization only in Table II.

TABLE II

| SAMPLE | PGA | GLY | PCL | CAP |
|---|---|---|---|---|
| 0 + 1 | 54.3 | 0.5 | 25.8 | 19.4 |
| 0 + 2 | 54.2 | 0.5 | 34.7 | 10.6 |
| 0 + 3 | 53.4 | 0.5 | 38.6 | 7.6 |
| 0 + 4 | 54.0 | 0.5 | 42.9 | 2.6 |
| 0 + 5 | 53.3 | 0.5 | 44.7 | 1.5 |
| 0 + 6 | 53.9 | 0.4 | 45.0 | 0.8 |

It is seen that the glycolide reacted quite rapidly, almost to completion after 1 hour. The slower reacting ε-caprolactone monomer reacted progressively throughout the reaction so that at 6 hours, the fraction of this monomer was down to 0.8 mole percent. It is apparent that the DEG initiated polymerization had a significantly higher reaction rate than the L-tartaric acid initiated reaction, even at a lower catalyst level than was used for the tartaric acid initiated reaction. The comparison is depicted graphically in FIG. 1, where unreacted ε-caprolactone monomer is plotted versus reaction time.

### Example 3

### Citric Acid as Initiator in the Synthesis of an AB-Type Block Copolymer of ε-Caprolactone And Glycolide

This example shows that lactone polymerization reactions that are initiated by tertiary alcohol initiators, catalyzed by tin catalysts such as stannous octoate, exhibit lower polymerization reaction rates than the reactions initiated by primary alcohol initiators, shown in Example 2.

The tertiary alcohol initiator of this example is citric acid. As seen herein, at equal reaction times, the tertiary alcohol initiator led to lower conversion of ε-caprolactone monomer into polymer than the primary alcohol type initiator.

“A” represents a randomized mid-block of 45/55 mole ratio of ε-caprolactone/glycolide prepolymer, and “B” represent a PGA block. The intended overall composition of the copolymer was 25 mole %, ε-caprolactone and 75 mole % PGA.

Into a conventional two gallon reactor provided with stirrer and jacket with heating medium was charged 1,481.2 grams (12.977 moles) of ε-caprolactone, 1,841 grams (15.861) moles of glycolide, 7.67 grams (0.03999 moles) of citric acid and 2.85 mls of a 0.33 molar solution of stannous octoate in toluene. In this example the molar ratio of all monomers (including the glycolide subsequently added in the second stage of the polymerization) to catalyst was 55,000:1. Into a separate melt-tank was charged 2,677.8 grams (23.07 moles) of glycolide. The reactor and the melt-tank were kept under 1 mm Hg vacuum for 20 minutes and the vacuum was released with nitrogen. The vacuum and nitrogen-breaking step was repeated. The reactor contents were heated by means of fluid circulation through the reactor jacket until the batch temperature reached 180° C. in about one hour. This was designated as “0” time. The reaction was continued for 7 additional hours at a heating fluid temperature of about 197° C. At this point glycolide that had been previously melted in the melt-tank was added to the reactor. The reaction continues at an approximate heating fluid temperature of 203° C. for 68 minutes and the product was discharged, cooled and dried in a tumble drier at room temperature and vacuum for 18 hours, followed by heating under vacuum for 24 hours and cooling.

During the course of the reaction, samples were taken from the reactor and were analyzed by NMR spectroscopy for monomer and polymer composition. Samples were designated as hours after “0” time (0+x) or as minutes after the second-stage glycolide transfer (T+y). The compositions are given on a molar basis. The data is displayed in Table III.

TABLE III

| Sample | PGA | GLY | PCL | Cap |
|---|---|---|---|---|
| 0 + 1 | 54.1 | 0.3 | 31.1 | 14.5 |
| 0 + 2 | 55.1 | 0.1 | 32.3 | 12.5 |
| 0 + 3 | 54.8 | 0.1 | 33.1 | 12.1 |
| 0 + 4 | 547 | 0.1 | 33.9 | 11.2 |
| 0 + 5 | 54.8 | 0.1 | 36.9 | 8.2 |
| 0 + 6 | 55.4 | 0.1 | 36.5 | 7.9 |
| 0 + 7 | 55.4 | 0.2 | 36.5 | 8.0 |
| T + 65 | 72.8 | 1.7 | 20.6 | 4.9 |
| Drop (T + 68) | 74.2 | 1.0 | 20.2 | 4.5 |

The dried sample had: 79.7% PGA, 0.4% GLY, 19.9% PCL, and 0% Cap. The example showed good reaction of glycolide. However, the ε-caprolactone reaction rate for the citric acid initiated polymerization was considerably slower than that in Example 2, where an initiator having a primary alcohol (DEG) was used.

### Example 4

### Glycolic Acid as Initiator in the Synthesis of an AB-Type Block Copolymer of ε-Caprolactone and Glycolide

This example shows that lactone polymerization reactions that are initiated by alcohol initiators that contain carboxylic acid groups, catalyzed by tin catalysts such as stannous octoate, result in polylactones that exhibit faster rate of hydrolysis, compared to alcohol initiators that do not contain carboxylic acid groups. As seen herein, the faster rate of hydrolysis is manifested in monofilaments that show lower in vitro breaking strength retention.

“A” represents a randomized mid-block of 45/55 mole ratio of ε-caprolactone/glycolide prepolymer, and “B” represents a PGA block. The intended overall composition of the copolymer is 25 mole %, ε-caprolactone and 75 mole % PGA.

Into a conventional two gallon reactor provided with stirrer and jacket with heating medium was charged 1481.2 grams (12.977 moles) of ε-caprolactone, 1841 grams (15.861) moles of glycolide, 3.95 grams (0.0519 moles) of glycolic acid and 3.14 mls of a 0.33 molar solution of stannous octoate in toluene. In this example the molar ratio of all monomers (including the glycolide subsequently added in the second stage of the polymerization) to catalyst was 50,000:1. Into a separate melt-tank was charged 2677.8 grams (23.07 moles) of glycolide. The reactor and the melt-tank were kept under 1 mm Hg vacuum for 20 minutes and the vacuum was released with nitrogen. The vacuum and nitrogen-breaking step was repeated. The reactor contents were heated by means of fluid circulation through the reactor jacket until the batch temperature reached 180° C. in about one hour. This was designated as “0” time. The reaction was continued for 6 additional hours at a heating fluid temperature of about 197° C. At this point glycolide that has been previously melted in the melt-tank was added to the reactor. The reaction was continued at an approximate heating fluid temperature of 203° C. for 68 minutes and the product was discharged, cooled and dried in a tumble drier at room temperature and vacuum for 18 hours, followed by heating under vacuum for 24 hours and cooling.

Both the undried and the dried polymer were analyzed by NMR spectroscopy for monomer and polymer composition. The results are given on a molar basis in Table IV.

TABLE IV

| Sample | PGA | GLY | PCL | Cap |
|---|---|---|---|---|
| Undried | 75 | 0.7 | 20.5 | 3.9 |
| Dried | 78.4 | 0.1 | 21.5 | 0.0 |

The dried polymer had an inherent viscosity of 1.5 dl/g as measured on a 0.1 g/dL solution in hexafluoroisopropanol, HFIP.

### Example 5

### Extrusion of the Polymer of Example 4

The polymer of Example 4 was extruded into size 3/0 monofilament sutures with a 0.625 inch Randcastle extruder, with an L/D of 24/1. The die hole was 0.034″. The extrudate was quenched in a water bath and was oriented by means of three godets and an air oven located between godet 2 and godet 3, under the conditions shown in Table V.

TABLE V

| Extrusion Conditions | Values |
|---|---|
| Extruder | |
| Barrel Pressure, psi | 400 |
| Screw RPM | 7.5 |
| Temperature | |
| Adapter, ° C. | 53 |
| Barrel 1, ° C. | 221 |
| Barrel 2, ° C. | 221 |
| Barrel 3, ° C. | 221 |
| Die, ° C. | 232 |
| Quench water, ° C. | 20 |
| Air oven, ° C. | 138 |
| Quench Tank | |
| Air gap, in. | 0.5 |
| Speed | |
| Godet #1, fpm | 10 |
| Godet #2, fpm | 62 |
| Godet #3, fpm | 62.5 |

The monofilament was annealed in an oven, under nitrogen for 6 hours at a temperature of 105° C. The annealed fiber had a diameter of 10.89 mils, a tensile strength of 10.81 pounds, an elongation at break of 39.54%, a Young's modulus of 206.4 kpsi and a knot strength of 7.44 pounds.

TABLE VI

| Monofilament | | | | | | | | | % BSR |
|---|---|---|---|---|---|---|---|---|---|
| Source | 0 day | | 2 days | | 4 days | | 7days | | at 7 days |
| Example 4 | 10.81 | lbs | 8.68 | lbs | 6.72 | lbs | 3.21 | lbs | 29.69% |
| DEG initiator | 11.8 | lbs | 10.1 | lbs | 7.9 | lbs | 4.5 | lbs | 38.1% |

The in vitro tensile strength in pounds of the monofilament of Example 5 [prepared from the polymer of Example 4] were determined initially (at day “zero”) and at 2, 4, and 7 days of incubation in bottles containing phosphate buffer at a pH of 7.27 and are compared in Table VI with typical properties of a monofilament from polymers initiated with diethylene glycol, DEG, at overall composition of 25 mole % PCL and 75 mole % PGA. The in vitro bath temperature was 40.9° C.; the buffer was based on sodium phosphate and potassium phosphate.

The last column of Table VI gives the percent of the original breaking strength remaining after 7 days in vitro. It is clear that example 4 gives a significantly lower BSR than the typical DEG initiated polymers. This is a reflection of the faster rate of hydrolysis of polymers initiated from an alcohol containing a carboxylic acid group.

### Example 6

### Evaluation of the Fiber of Example 5

A hydrolysis profile was also obtained for the suture of Example 5 and was compared with a typical hydrolysis profile of a monofilament from a polymer initiated with DEG at overall composition of 25 mole % PCL and 75 mole % PGA. Test specimens were hydrolytically degraded at 75° C.±0.2° C. while maintaining a constant pH of 7.27 by titrating with a standard base (NaOH 0.05N) and measuring the volume, V(t) of base used versus time, by means of an automatic titrator. From an analysis of the volume, V(t), versus time curves, the time in hours required to obtain 90% hydrolysis is determined. The faster hydrolysis is reflected in the lower times required to achieve a given percentage hydrolysis. It was found that for monofilament of example 4, the average time required to achieve 90% hydrolysis was 46 hours (n=3). In comparison, for monofilaments from a polymer initiated with DEG at overall composition of 25 mole % PCL and 75 mole % PGA, the average time to achieve 90% conversion was 62.3 hours.

This is a reflection of the faster rate of hydrolysis of polymers initiated from an alcohol containing a carboxylic acid group.

### Example 7

### 1,3-Dihydroxy-2,2-Dicarboxypropane As Initiator in the Synthesis of an ABA-Type Block Copolymer of ε-Caprolactone and Glycolide

Into a conventional two gallon reactor provided with stirrer and jacket with heating medium is charged 1,234.3 grams (10.814 moles) of ε-caprolactone, 1,534.2 grams (13.217 moles) of glycolide, 5.4605 grams (0.033274 moles) of 1,3-dihydroxy-2,2-dicarboxypropane and 2.38 mls of a 0.33 molar solution of stannous octoate in toluene. In this example the molar ratio of all monomers (including the glycolide subsequently added in the second stage of the polymerization) to catalyst was 55,000:1. Into a separate melt tank is charged 2,231.5 grams (19.225 moles) of glycolide (second stage). The reactor and the melt tank are kept under 1 mm hg vacuum for 20 minutes and the vacuum is released with nitrogen. The vacuum and nitrogen-breaking step is repeated. The reactor contents are heated by means of fluid circulation through the reactor jacket until the batch temperature reaches 180° C. in about one hour. This is designated as “0” time. The reaction is continued for 6 additional hours at a heating fluid temperature of about 197° C. At this point glycolide that has been previously melted in a separate melt tank was added to the reactor at a controlled rate. The reaction is continued at an approximate heating fluid temperature of 203° C. for 75 minutes and the product is discharged, cooled and dried in a tumble drier at room temperature and vacuum for 18 hours, followed by heating under vacuum for 24 hours and cooling.

The 1,3-dihydroxy-2,2-dicarboxypropane initiated polymerization has a significantly higher reaction rate than the L-tartaric acid initiated reaction. A polymer can be made using as an initiator the monol, 1-hydroxy-2,2,2-trimethcarboxyethane.

The novel polymers of the present invention may be melt processed by conventional means into numerous useful products. They include monofilament sutures of the traditional un-barbed variety, as well as barbed monofilament sutures; multifilament sutures; injection molded products, such as clips staples and straps; films, etc.

The novel products of the present invention made from the inventive polymers exhibit a faster loss of mechanical properties post-implantation than currently available products of the same composition but made from conventional polymerization initiators.

The inventive products made from the inventive polymers exhibit a faster absorption rate than currently available products of the same composition but made from conventional polymerization initiators.

In general the novel polymers of the present invention exhibit molecular weights suitable to support high mechanical properties. They would necessarily need to be higher than those molecular weights generally employed in coatings having fast absorption rates.

Although this invention has been shown and described with respect to detailed embodiments thereof, it will be understood by those skilled in the art that various changes in form and detail thereof may be made without departing from the spirit and scope of the claimed invention.

We claim:

1. A polymerization process for making an absorbable polylactone polymer, comprising:
providing a lactone monomer selected from the group consisting of glycolide, L(−)-lactide, D(+)-lactide, meso-lactide, 1,4-dioxanone, ε-caprolactone, or trimethylenecarbonate;
combining the lactone monomer with a polymerization initiator and a catalyst; and,
polymerizing the lactone monomer in the presence of the polymerization initiator, thereby providing a linear absorbable polylactone polymer,
wherein the polymerization initiator comprises a monol or diol that contains at least one primary alcohol group and also contains at least two carboxylic acid groups.

2. The process of claim 1, wherein a molar ratio of monomer-to-initiator is from about 300:1 to about 50,000:1.

3. The process of claim 1, wherein the initiator comprises two or more carboxylic acid groups and is a monol, C₄ or higher, primary aliphatic alcohol.

4. The process of claim 3, wherein the monol initiator is 1-hydroxy-2,2,2-trimethcarboxyethane.

5. The process of claim 1, wherein the initiator is selected from the group consisting of C₄H₆O₅, HOOC—CH(CH₂OH)—COOH; C₅H₈O₅, HOOC—C(CH₃)(CH₂OH)—COOH; C₇H₁₂O₅, HOOCCH₂—C(CH₃)(CH₂OH)—CH₂COOH; C₉H₁₆O₅, HOOCCH₂—C(CH₂CH₃)(CH₂CH₂OH)—CH₂COOH; C₅H₈O₆, HOOC—C(CH₂OH)₂—COOH; C₇H₁₂O₆, HOOCCH₂—C(CH₂OH)₂—CH₂COOH; C₈H₁₄O₆, HOOCCH₂—C(CH₂OH)(CH₂CH₂OH)—CH₂COOH; C₉H₁₆O₆, HOOCCH₂—C(CH₂CH₂OH)₂—CH₂COOH; C₅H₆O₇, HOCH₂—C—(COOH)₃; C₇H₁₀O₇, HOCH₂—C(CH₂COOH)₂—COOH; C₈H₁₂O₇, HOCH₂—C—(CH₂COOH)₃; and C₉H₁₄O₇, HOCH₂CH₂—C—(CH₂COOH)₃.

6. An absorbable, linear polylactone polymer made by the process of claim 1.

7. The absorbable polymer of claim 6, wherein the polymer is melt processable.

8. The absorbable polymer of claim 6, having an Mn of about 17,000 to about 100,000 Daltons.

9. A medical device comprising the polymer of claim 6.

10. The medical device of claim 9 comprising a device selected from the group consisting of sutures, clips, staples, tacks, surgical fasteners, meshes, fabrics, and fibers.

11. The medical device of claim 9, wherein the device is capable of being deformed.

12. The medical device of claim 9, wherein the device is resiliently deformable.

13. The process of claim 1, wherein the initiator possesses two or more carboxylic acid groups and is a diol, C₄ or higher, having at least one primary aliphatic alcohol.

14. The process of claim 13, wherein the diol initiator is 1,3-dihydroxy-2,2-dicarboxypropane.

15. An absorbable, linear polylactone polymer made by the process of claim 5.

16. The medical device of claim 10, wherein the device is a suture.

17. The medical device of claim 16, wherein the suture is a multifilament suture.

18. The medical device of claim 16, wherein the suture is a monofilament suture.

19. A substantially linear aliphatic absorbable polyester comprising a monovalent unit of formula I:
—O—CH₂—R₁ I
and divalent repeating units selected from the group of formulae consisting of:
[—O—CH₂—C(O)—]ₐ II
[—O—C(H)(CH₃)—C(O)—]_{b} III
[—O—CH₂CH₂—O—CH₂CH₂—C(O)—]_{c} IV
[—O—CH₂CH₂CH₂CH₂CH₂—C(O)—]_{d} V
[—O—CH₂CH₂CH₂—O—C(O)—]ₑ VI
and combinations thereof, wherein R₁ is an alkyl group containing two or more carboxylic acid groups, and a, b, c, d, and e are integers such that a weight average molecular weight of said substantially linear aliphatic absorbable polyester is between about 35,000 Daltons and 200,000 Daltons.

20. A medical device comprising the polymer of claim 19.

21. The medical device of claim 20 comprising a device selected from the group consisting of sutures, clips, staples, tacks, surgical fasteners, meshes, fabrics, and fibers.

22. The medical device of claim 20, wherein the device is capable of being deformed.

23. The medical device of claim 20, wherein the device is resiliently deformable.

24. The medical device of claim 21, wherein the device is a suture.

25. The medical device of claim 24, wherein the suture is a multifilament suture.

26. The medical device of claim 24, wherein the suture is a monofilament suture.

27. A substantially linear aliphatic absorbable polyester comprising a first divalent unit of formula IA:
[—O—C(R₂)(R₃)—R₄—CH₂—O—] IA
and divalent repeating units selected from the group of formulae consisting of:
[—O—CH₂—C(O)—]ₐ IIA
[—O—C(H)(CH₃)—C(O)—]_{b} IIIA
[—O—CH₂CH₂—O—CH₂CH₂—C(O)—]_{c} IVA
[—O—CH₂CH₂CH₂CH₂CH₂—C(O)—]_{d} VA
[—O—CH₂CH₂CH₂—O—C(O)—]ₑ VIA
and combinations thereof, wherein R₂ and R₃ are independently hydrogen or an alkyl group containing 1 to 8 carbon atoms, R₄ is an alkyl group containing two or more carboxylic acid groups, and a, b, c, d, and e are integers such that a weight average molecular weight of said substantially linear aliphatic absorbable polyester is between about 35,000 Daltons and 200,000 Daltons.

28. A medical device comprising the polymer of claim 27.

29. The medical device of claim 28 comprising a device selected from the group consisting of sutures, clips, staples, tacks, surgical fasteners, meshes, fabrics, and fibers.

30. The medical device of claim 28, wherein the device is capable of being deformed.

31. The medical device of claim 28, wherein the device is resiliently deformable.

32. The medical device of claim 29, wherein the device is a suture.

33. The medical device of claim 29, wherein the suture is a multifilament suture.

34. The medical device of claim 29, wherein the suture is a monofilament suture.
